(19) United States
(12) Patent Application Publication
(10) Pub. No.: US 2021/0113652 A1
(43) Pub. Date: Apr. 22, 2021

(54) MASPIN-BASED INHIBITION OF OSTEOCLAST ACTIVITY AND PROMOTION OF BONE FORMATION

(71) Applicant: Northwestern University, Evanston, IL (US)

(72) Inventor: Ming Zhang, Glencoe, IL (US)

(21) Appl. No.: 16/887,509

(22) Filed: May 29, 2020

Related U.S. Application Data
(63) Continuation of application No. 15/588,179, filed on May 5, 2017, now abandoned.
(60) Provisional application No. 62/332,064, filed on May 5, 2016.

(51) Int. Cl. A61K 38/16; A61K 38/57

(52) U.S. Cl. CPC A61K 38/16; A61K 31/00; A61K 38/57

(57) ABSTRACT

Provided herein are compositions and methods for the treatment of bone disorders through the maspin-based inhibition of osteoclastogenesis and osteoclast activity and the promotion of bone formation.

### CROSS-REFERENCE TO RELATED APPLICATIONS

The present application is a continuation of U.S. patent application Ser. No. 15/588,179, filed May 5, 2017, which claims the priority benefit of U.S. Provisional Patent Application 62/332,064, filed May 5, 2016, each of which is incorporated by reference in its entirety.

### STATEMENT REGARDING FEDERAL FUNDING

This invention was made with government support under CA079736 awarded by the National Institutes of Health. The government has certain rights in the invention.

### SEQUENCE LISTING

The text of the computer readable sequence listing filed herewith, titled “34828-303 SEQUENCE LISTING ST25”, created Jan. 8, 2021, having a file size of 4,000 bytes, is hereby incorporated by reference in its entirety.

### FIELD

Provided herein are compositions and methods for the treatment of bone disorders through the maspin-based inhibition of osteoclastogenesis and osteoclast activity and the promotion of bone formation.

### BACKGROUND

It is estimated that over 200 million people worldwide suffer from osteoporosis, and many more patients suffer from defects in the balance between bone deposition and bone resorption. There are two existing osteoporosis products on the market. One is the highly potent anti-osteoclastic agent Denosumab (anti-RANKL monoclonal antibody), and the other is the general inhibitor bisphosphate. Denosumab is very potent but expensive. Bisphosphate drug is not very potent but is widely accessible. Both drugs share some common adverse effects in increased risks of infections, including but not limited to: cellulitis, hypocalcemia, osteonecrosis (jaw and hip), and skin eczema. Improved therapeutics for osteoporosis, as well as other bone disorders, are needed.

Maspin (mammary serine protease inhibitor) is a protein that in humans is encoded by the SERPINB5 gene. Maspin belongs to the serpin (serine protease inhibitor) superfamily (Khalkhali-Eilis, Clin. Cancer Res. (2006) 12 (24): 7279-83; incorporated by reference in its entirety). SERPINB5 was originally reported to function as a tumor suppressor gene in epithelial cells, suppressing the ability of cancer cells to invade and metastasize to other tissues (Zou et al. Science (1994) 263 (5146): 526-9; incorporated by reference in its entirety). Maspin is naturally-expressed in bone osteoblasts and other epithelial cells. Overexpression of maspin does not have any observable toxic side effects in physiological tests in vivo (Shi et al., Molecular Therapy (2002) 5, 755-761; incorporated by reference in its entirety).

### SUMMARY

Provided herein are compositions and methods for the treatment of bone disorders through the maspin-based inhibition of osteoclastogenesis and osteoclast activity and the promotion of bone formation.

In some embodiments, provided herein are methods of treating a bone disorder in a subject comprising administering to the subject an agent that enhances maspin expression, level, and/or activity within the subject. In some embodiments, the agent is formulated in a pharmaceutical composition. In some embodiments, the agent is a maspin peptide or polypeptide comprising at least 60% (e.g., 60%, 65%, 70%, 75%, 80%, 85%, 90%, 95%, 100%, or ranges therebetween) sequence identity or similarity to all or a portion of SEQ ID NO: 1. In some embodiments, the agent is a maspin peptide or polypeptide with less than 100% sequence identity to SEQ ID NO: 1. In some embodiments, the agent interacts with the cellular machinery to increase expression of maspin within the cells of the subject. In some embodiments, expression is increased within osteoblast cells. In some embodiments, the agent is a nucleic acid. In some embodiments, the nucleic acid encodes a maspin peptide or polypeptide and sequences to facilitate expression within the cells of the subject. In some embodiments, the nucleic acid inhibits expression of an inhibitor of maspin activity or expression (e.g., by antisense or RNA interference). In some embodiments, the nucleic acid alters the genomic DNA of the cells of the subject to enhance expression of the subject's own maspin (e.g., CRISPR). In some embodiments, the bone disorder is selected from the group consisting of osteopenia, osteoporosis, osteomalacia, rachitis, osteitis fibrosa, aplastic bone diseases, metabolic bone diseases, cancer-induced osteolytic lesions, osteolysis, leucopenia, bone malformation, hypercalcemia, and nerve compression syndrome. In some embodiments, the agent is administered locally to a treatment site or systemically to the subject.

In some embodiments, provided herein are methods of facilitating/promoting bone growth (or inhibiting bone loss) in a subject comprising increasing the level of maspin and/or maspin-based peptides or polypeptides within the subject. In some embodiments, bone growth is facilitated by inhibiting osteoclastogenesis and/or osteoclast activity. In some embodiments, bone growth is facilitated by reducing the rate of bone resorption. In some embodiments, the level of maspin and/or maspin-based peptides or polypeptides is increased within the subject by administering maspin and/or maspin-based peptides or polypeptides to the subject. In some embodiments, the level of maspin and/or maspin-based peptides or polypeptides in increased within the subject by enhancing expression of endogenous maspin within the subject's cells. In some embodiments, the level of maspin and/or maspin-based peptides or polypeptides in increased within the subject by administering cells to a subject that express maspin and/or maspin-based peptides or polypeptides.

In some embodiments, provided herein are pharmaceutical compositions comprising an active agent that enhances expression, level, or activity of maspin within a subject. In some embodiments, provided herein is the use of a maspin peptide/polypeptide or an enhancer of maspin expression or activity to treat a bone disorder, stimulate bone repair, and/or promote bone growth.

In some embodiments, compositions and methods are provided herein for the inhibition of osteoclast formation through inhibiting expression of key genes responsible for osteoclastic activity, such as AV integrin, TRAP, cathepsin K, GTPase Rac1, etc. In some embodiments, compositions and methods are provided herein for the inhibition of osteoclast actin ring formation.

### BRIEF DESCRIPTION OF THE DRAWINGS

FIG. 1. Maspin inhibits osteolytic bone degradation in tumor-inoculated BALB/c mice. Panel A-B: Representative X-ray radiographs of mouse tibiae inoculated with mammary tumor cells TM40D (Panel A) or maspin-expressing TM40D-Mp tumor cells (Panel B), at the end point of 4 weeks following tumor inoculation. Note that mice inoculated with TM40D tumors display severe osteolytic lesion comparing to that with TM40D-Mp cells. Panel E: Summary of osteolytic lesion data (radiography analysis) in 20 mice (10 mice/group) at 2-week and 4-week time points. Panel C: Histological analysis of bone section of tumor-inoculated tibiae by H&E staining. Arrow heads indicate osteoclastic cells. Panel D: TRAP (tartrate-resistant acid phosphatase) staining of osteoclastic cells in bone sections. Arrows indicate osteoclastic cells. Panel F: Summary of TRAP staining data of osteoclasts in mouse tibiae inoculated with TM40D or TM40D-Mp cells. Mice inoculated with maspin-expressing TM40D-Mp tumor cells have significantly decreased osteoclast numbers than that with TM40D tumor cells.

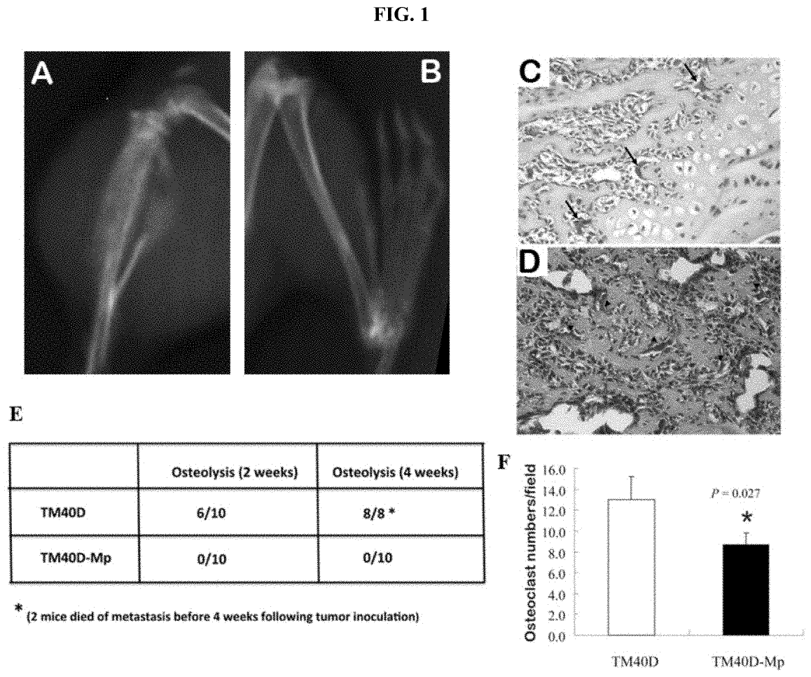

FIG. 2. Maspin inhibits osteoclast formation in mouse primary bone marrow co-culture assays. Osteoclastogenesis analyses of primary bone marrow cells co-cultured with breast cancer cells with or without maspin expression. (Panel A) Newly isolated primary bone marrow cells only (control). (Panel B) Bone marrow cells co-cultured with mammary tumor TM40D cells. (Panel C) Bone marrow cells co-cultured with maspin-expressing TM40D-Mp cells. (Panel D) Bone marrow cells co-cultured with TM40D-Mp cells and rabbit anti-maspin antibody. (Panel E) Bone marrow cells co-cultured with TM40D-Mp cells and rabbit IgG. (F) Summary of percentage of osteoclasts in co-cultured cells analyzed by TRAP staining. TRAP-positive cells containing 2 or more nuclei are defined as osteoclasts.

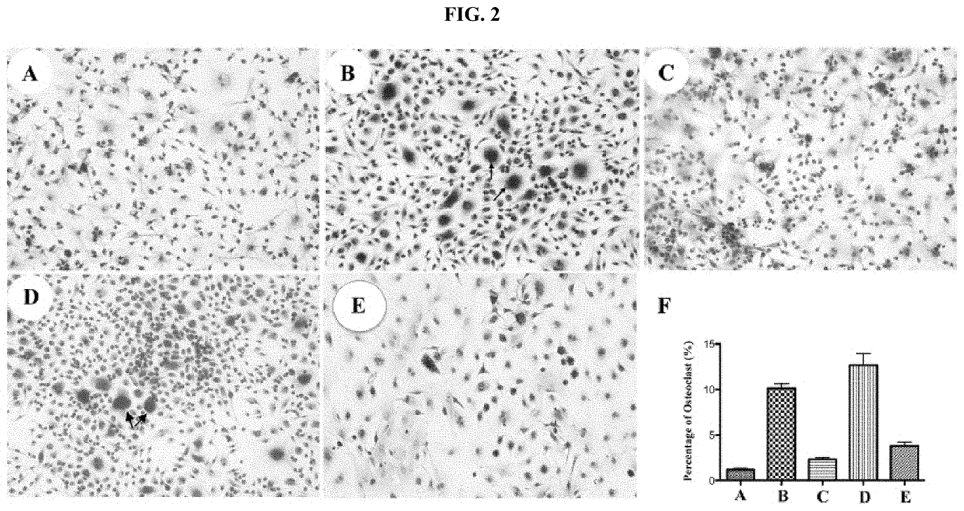

FIG. 3. Recombinant maspin protein inhibits osteoclast formation in the differentiation assay of pre-osteoclastic RAW246.7 cells. RAW246.7 cells were treated with RANKL (50 -100 ng/ml) and different doses of maspin for 8 days, and cell cultures were used for TRAP staining. Note that RANKL stimulates the differentiation of RAW246.7 to form osteoclasts, but maspin at the tested dosages (0.1-10 ug/ml) completely inhibits osteoclast formation under RANKL stimulation.

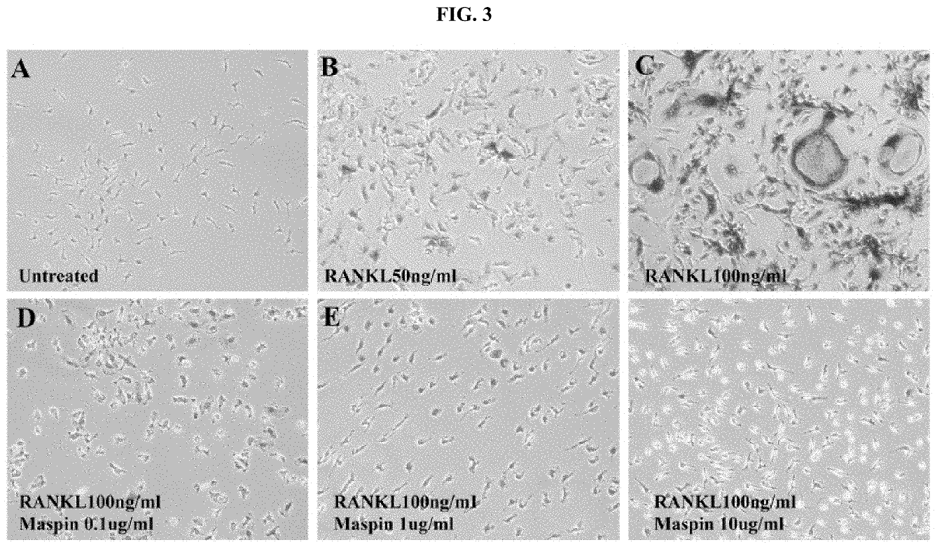

FIG. 4. Maspin inhibits osteoclast formation through inhibiting expression of key genes responsible for osteoclastic activity, including AV integrin, TRAP, and cathepsin K. Cells were stimulated by RANKL at 50 ng/ml and cultured with Gst control or Gst-maspin for 3 days or 6 days. Lane 1, Raw 246.7 cells untreated at day 1; Lane 2, Cells treated by Gst control for 3 days; Lane 3, Cells treated by Gst-maspin for 3 days; Lane 4, Cells treated by Gst control for 6 days; Lane 5, Cells treated by Gst-maspin for 3 days. Note that Gst-maspin significantly inhibits gene expression of Av integrin, TRAP, and cathepsin K in RAW246.7 cells on both day 3 (lane 3) and day 6 (lane 5).

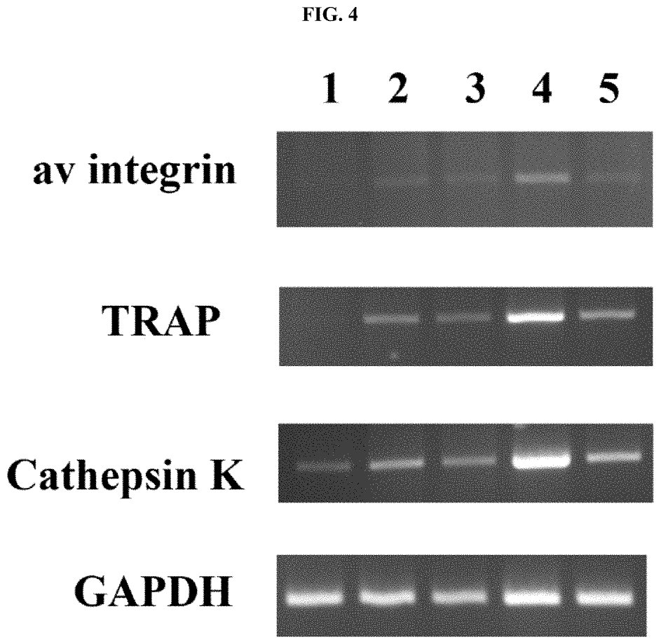

FIG. 5. Maspin inhibits small GTPase Rac1 activity in osteoclastic cells. RAW246.7 cells were cultured with RANKL (50 ng/ml) for 6 days to form osteoclasts. Cells were then washed with cold PBS and serum starved for three hours before they were supplied with RANKL and new cell culture medium containing either Gst or GST-Maspin for 1 to 3 hours. Cells were then harvested for extracts. Active Rac1 protein was pull down from cell extracts using Gst-Pakl-PBD and Western blot analysis was performed to analyze level of total Rac1 and active Rac1 in Gst or Gst-maspin treated cells. Experiments were from four independent assays and relative active Rac1 levels were done comparing active and total Rac1 at each time point. Cells treated with Gst-maspin at 3 hours had significantly reduced active Rac1 level compared to the control treated cells.

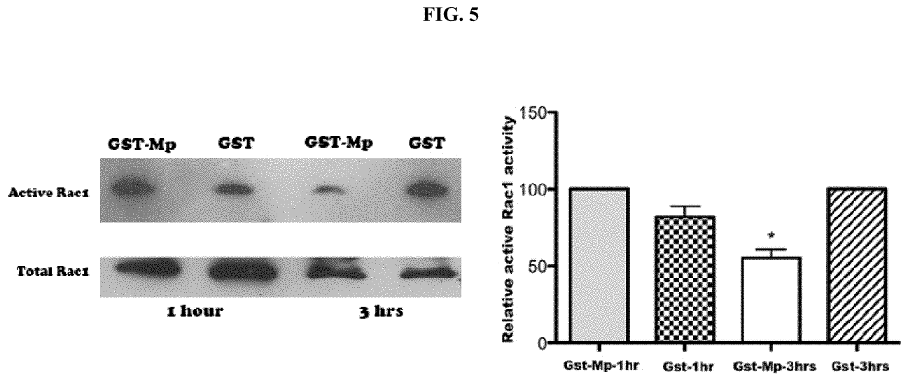

FIG. 6. Panels A-C: Maspin inhibits osteoclast actin ring formation. Osteoclasts display osteolytic activity by forming actin rings. RAW246.7 were pre-cultured with RANKL (50 ng/ml) for 8 days to form mature osteoclasts. Cells were first washed with cold PBS twice to disrupt actin rings, warm medium with RANKL were added to cell culture with or without Gst or GST-maspin and cells were then cultured for 2 hours at 37° C. Cells were then washed and stained with phalloidin for actin ring formation. Cells with strong and clear actin ring belts were counted as positive. Five fields of cell staining were counted for each sample and experiments repeated three times. The percentage of cells forming strong and clear actin rings was calculated. Maspin treated cells have significant reduction in the numbers of actin ring forming osteoclasts (53%) compared to control or Gst-treated cells (89.5%).

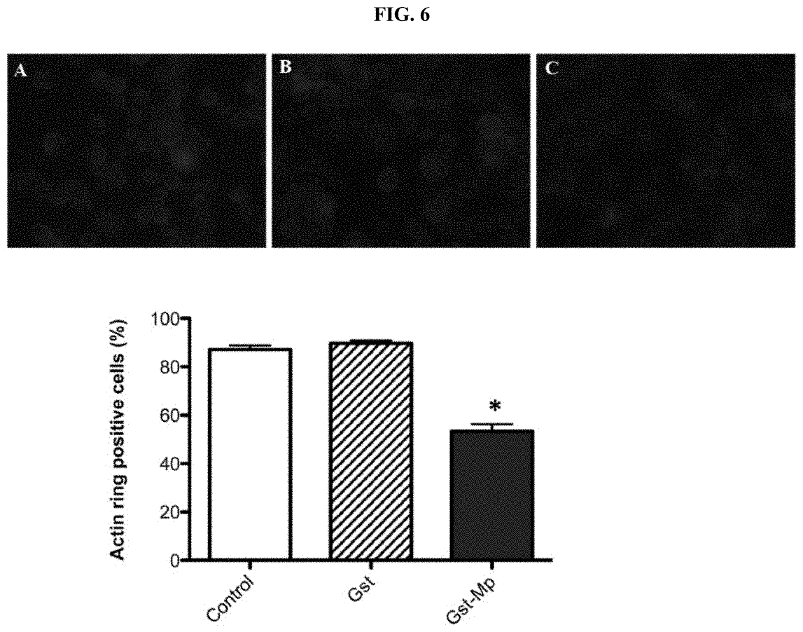

FIG. 7. Maspin promotes new bone formation in an organ culture model. Panel A, calvariae were removed from 4 days-old BABL/c new-born mice and cultured in an ex vivo organ culture medium. They were either treated with or without concentrated conditional medium from mouse mammary tumor TM40D cells or maspin-expressing TM40D-Mp cells or with recombinant maspin (GST-Mp) with or without an anti-maspin antibody for 7 days. H&E staining of representative images were shown. Arrows indicate osteoblast cells. Arrowhead indicates new bone area. Panel B, quantitation of the average number of osteoblasts under different treatments listed in Panel A.

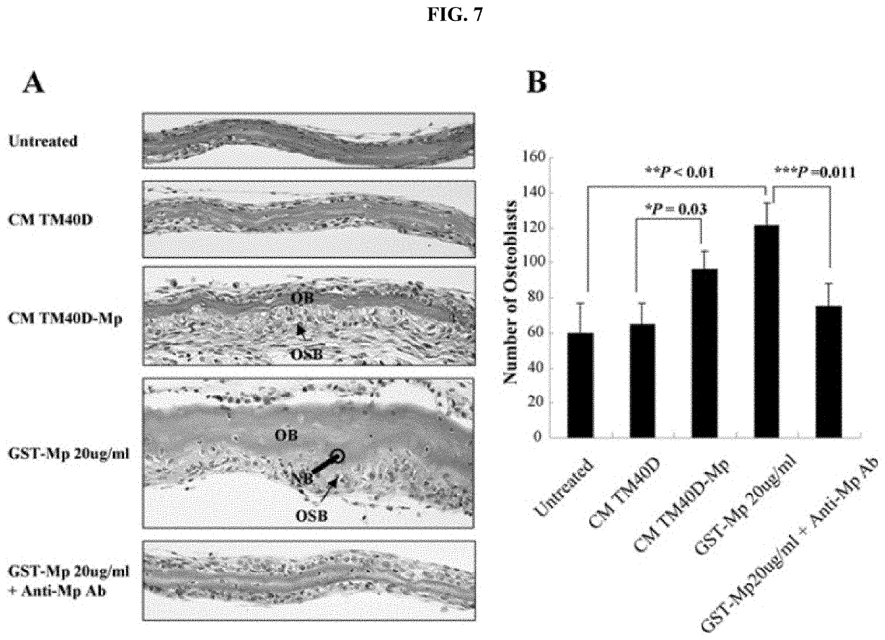

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a maspin peptide” is a reference to one or more maspin peptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language; such embodiments encompass multiple closed “consisting of” and/or “consisting essentially of” embodiments, which may alternatively be claimed or described using such language.

As used herein, the term “subject” broadly refers to any animal, including but not limited to, human and non-human mammals (e.g., primates, rodents, dogs, cats, cows, horses, sheep, etc.). As used herein, the term “patient” typically refers to a subject that is suffering from and/or being treated for a disease or condition (e.g., osteoporosis, etc.).

As used herein, the terms “bone density” and “bone mineral density” (“BMD”) refer to the amount of bone mineral present in bone tissue (i.e., mass of bone mineral per volume of bone). Bone density is typically measured clinically by proxy according to optical density per bone surface area upon imaging. A bone mineral density test yields a T-score, which is a measure of the tested density versus that of a healthy thirty year old. According to World Health Organization criteria, a T-score of −1.0 or higher is a normal BMD, a T-score of −1.0 to −2.5 (or 1 to 2.5 standard deviations below the mean of a thirty-year-old man/woman) is diagnostic of osteopenia, and a T T-score of below −2.5 (or greater that 2.5 standard deviations below the mean of a thirty-year-old man/woman) is diagnostic of osteoporosis.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 25 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide or nucleic acid is one comprising a non-natural sequence (e.g., a peptide without 100% identity with a naturally-occurring protein or a fragment thereof).

As used herein, the term “peptoid” refers to a class of peptidomimetics where the side chains are functionalized on the nitrogen atom of the peptide backbone rather than to the α-carbon.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R));

polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence “having at least Y% sequence identity with SEQ ID NO:Z” may have up to X substitutions relative to SEQ ID NO:Z, and may therefore also be expressed as “having X or fewer substitutions relative to SEQ ID NO:Z.”

As used herein, the term “wild-type,” refers to a gene or gene product (e.g., protein) that has the characteristics (e.g., sequence) of that gene or gene product isolated from a naturally occurring source, and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature, but have altered sequences when compared to the wild-type gene or gene product; they are not the most commonly occurring sequence. “Synthetic mutants” are genes or gene products that have altered sequences when compared to the wild-type gene or gene product and do not occur in nature. Mutant genes or gene products may be naturally occurring sequences that are present in nature, but not the most common variant of the gene or gene product, or “synthetic,” produced by human or experimental intervention.

The term “effective dose” or “effective amount” refers to an amount of an agent which results in a desired biological outcome (e.g., inhibition of osteoclast production and/or activity)

As used herein, the terms “administration” and “administering” refer to the act of providing a therapeutic, prophylactic, or other agent to a subject for the treatment or prevention of one or more diseases or conditions. Exemplary routes of administration to the human body are through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the term “treat,” and linguistic variations thereof, encompasses therapeutic measures, while the term “prevent” and linguistic variations thereof, encompasses prophylactic measures, unless otherwise indicated (e.g., explicitly or by context).

As used herein, the terms “co-administration” and “co-administering” refer to the administration of at least two agent(s) or therapies to a subject (e.g., a maspin-based agent and one or more additional therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent (e.g., a maspin-based agent) with a carrier, inert or active, making the composition especially suitable for diagnostic or therapeutic use in vitro, in vivo or ex vivo.

The term “pharmaceutically acceptable” as used herein, refers to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

### DETAILED DESCRIPTION

Provided herein are compositions and methods for the treatment of bone disorders through the maspin-based inhibition of osteoclastogenesis and osteoclast activity and the promotion of bone formation.

Maspin is well known for its inhibitory role in cancer progression. Experiments conducted during development of embodiments herein demonstrate that maspin effectively blocks osteoclast cell formation induced by RANKL ligand. Maspin also inhibits mature osteoclast activity. Initial experiments demonstrated that maspin blocks tumor-induced osteolytic degradation of the bone. Further studies revealed that maspin actively blocks osteoclast formation in RANKL-induced in vitro cell culture and mouse primary bone marrow cell culture. Specifically, maspin inhibits the expression of key osteoclastic genes, including av integrin, TRAP, and cathepsin K. In mature osteoclasts, maspin counteracts RANKL's effect and inhibits Rac1 activity of osteoclasts, and inhibits osteoclast actin ring formation. These findings demonstrate the usefulness of maspin as a therapeutic target against osteolytic bone degradation, as well as a drug promoting/assisting bone wound healing and new bone formation.

The findings herein demonstrate unique mechanisms for treating bone degradation diseases, such as osteoporosis, a pathological bone disease happens in aging people with excessive bone loss. Because bone is tissue under constant balancing actions between bone-degrading osteoclasts and bone-forming osteoblasts, inhibiting the formation and activity of osteoclasts will favor or promote osteoblastic activity, thereby promoting new bone formation and bone wound healing. Further, maspin is naturally produced by osteoblasts in the bone microenvironment. This represents a previously unappreciated and unexploited mechanism for osteoblast-produced maspin counteracting the effect of osteclastic activity. Experiments have demonstrated that long-term treatment with maspin in animals has no side effects. Therefore, maspin may be delivered to human patients as a non-toxic therapeutic product against various bone diseases.

In some embodiments, compositions and methods are provided herein for the treatment or prevention of bone diseases. In some embodiments, the bone disease or condition to be prevented or treated includes bone loss or damage due to cancer metastatic to the bone, bone wound (e.g., due to injury), post-surgical bone repair, osteopenia, osteoporosis, osteomalacia, rachitis, osteitis fibrosa, aplastic bone diseases, metabolic bone diseases, cancer-induced osteolytic lesions, osteolysis, leucopenia, bone malformation, hypercalcemia and nerve compression syndrome.

Bone tissue is dynamic: healthy bones require continuous formation and resorption to regulate mineral homeostasis, through the process of bone metabolism or bone remodeling. Bone is mainly composed of bone cells and bone matrix, and the bone cells comprise osteoblasts, osteoclasts, and osteocytes. Osteoblasts are derived from the differentiation of bone marrow mesenchymal stem cells and can secrete the bone matrix to be mineralized, so as to form new bone; the osteoclasts are derived from the differentiation of monocytes, and can perform bone resorption and decomposition and release minerals in the bone to blood; both osteoblasts and osteoclasts are useful for bone retention in the body.

An imbalance in bone formation and bone resorption may lead to bone disorders such as osteoporosis. Osteoporosis is a kind of metabolic bone disease characterized by reduced bone mass density. Aging and menopause are among the most importance risk factors for developing osteoporosis. In general, the bone mass density of human will gradually increase from birth, and during puberty, the bone mass density rises faster due to the effect of sexual hormones. To about 35 years of age, the bone mass density reaches a peak. After about 45 years old, the bone decomposition rate will be larger than the bone formation rate, resulting in gradual loss of bone mass and decreased bone mass density. Osteoporosis is defined as T value of the bone density below −2.5. Osteoporosis will make bone hollow and fragile and thus cause increased risk of fractures.

Osteoporosis is roughly divided into primary osteoporosis and secondary osteoporosis. Primary Osteoporosis can be further divided into two types: postmenopausal (Type I) osteoporosis and senile (Type II) osteoporosis. Type I osteoporosis occurs in postmenopausal women; Type II osteoporosis generally occurs in the elderly after the age of 70. Secondary Osteoporosis: it could be found in any age of the male or female and is caused by factors, such as hyperthyroidism, diabetes, genetic diseases, rheumatoid arthritis, and so on. 80 percent of osteoporosis belongs to the primary osteoporosis, and particularly to postmenopausal osteoporosis.

Factors linked to osteoporosis may include: (1) Aging: bone aging with ages and thus bone mass density dropping off; (2) Sex: the bone mass density of the female being less than that of the male; (3) Underweight: the bone mass density being relatively low for people with lighter weight; (4) Decreased estrogen: estrogen stimulating bone regeneration and inhibiting bone decomposition; (5) Diets: calcium, vitamin D, or protein deficiency; excessive smoking or drinking; drinking coffee or tea; in-taking high-protein or high-salt foods; (6) Irregular living habits: lack of exercise, less sun, prolonged bed rest; (7) Genetic causes: several members in a family having osteoporosis; and (8) Specific diseases: hyperthyroidism, gonadal insufficiency, rheumatoid arthritis, diabetes, liver disease patients being predisposed to get osteoporosis.

In the case of secondary osteoporosis, it may occur, even in young subjects, as the result of, for example, certain diseases or drugs. In such cases, bone strength becomes decreased and the probability of bone fracture increases, depending on severity of and exposure time to diseases or drugs. Such diseases include hyperthyroidism, hyperparathyroidism, Cushing's Syndrome (hypersecretion disease of andrenocorticotropic hormone), premature menopause, menopause due to artificial operation (oophorectomy), sexual dysfunction, chronic liver diseases (liver cirrhosis), rheumatoid arthritis, chronic kidney dysfunction and gastrectomy, and drugs such as steroids (corticosteroids), anticovulsant drugs (drugs for epilepsy), heparin, etc.

In some embodiments, provided herein are compositions (e.g. maspin, enhancers of maspin activity, etc.) methods for treating individuals that have diseases characterized by bone loss and/or inadequate bone formation. Maspin and/or maspin enhancers are administered in an amount effective to inhibit/reduce osteoclastogenesis and/or osteoclast function, and thereby reduce bone loss and/or promote bone regeneration.

In some embodiments, by inhibiting osteoclastogenesis and/or osteoclast function, bone erosion is prevented and bone loss is reduced. In some embodiments, by inhibiting osteoclastogenesis and/or osteoclast function, bone regeneration is enhanced and bone formation is increased. Patients suffering from diseases characterized by bone loss or some bone formation defects are treated by administering an effective amount of compositions to inhibit osteoclastogenesis and/or osteoclast function (e.g., maspin or enhancer of maspin activity or expression). In some embodiments, patients identified as being susceptible to diseases characterized by bone loss are prophylactically treated by administering an effective amount of compositions to inhibit osteoclastogenesis and/or osteoclast function (e.g., maspin or enhancer of maspin activity or expression).

In some embodiments, individuals with a disease characterized by bone loss are identified by those having ordinary skill in the art by well-known diagnostic means and criteria. In some embodiments, individuals with a disease characterized by inadequate bone formation are identified by those having ordinary skill in the art by well-known diagnostic means and criteria. In some embodiments, individuals who are susceptible to a disease characterized by bone loss are identified by those having ordinary skill in the art, for example, based upon personal/family medical history, diagnostic criteria, and/or the presence of genetic markers or genes associated with a disease characterized by bone loss.

In some embodiments, provided herein are pharmaceutical compositions and/or therapeutic agents that inhibit osteoclastogenesis and/or osteoclast function via a maspin-based mechanism, and methods of treating conditions and diseases related to bone loss or inadequate bone formation therewith. In some embodiments, a pharmaceutical compositions and/or therapeutic agents comprises a full-length maspin (e.g., wild-type, natural variant, synthetic/artificial variant, etc.). In some embodiments, a pharmaceutical compositions and/or therapeutic agent comprises a maspin peptide (e.g., a fragment of a full-length maspin (e.g., wild-type, natural variant thereof) or a synthetic/artificial variant thereof.

In some embodiments, a therapeutic agent within the scope herein is a full-length maspin. For example, in some embodiments, a therapeutic agent comprises full-length, wild-type human maspin (SEQ ID NO: 1):

| | |
|---|---|
| 1 | mdalqlansa favdlfkqlc ekeplgnvlf spiclstsls laqvgakgdt aneigqvlhf |
| 61 | envkdipfgf qtvtsdvnkl ssfyslklik rlyvdkslnl stefisstkr pyakeletvd |
| 121 | fkdkleetkg qinnsikdlt dghfenilad nsvndqtkil vvnaayfvgk wmkkfpeset |
| 181 | kecpfrlnkt dtkpvqmmnm eatfcmgnid sinckiielp fqnkhlsmfi llpkdvedes |
| 241 | tglekiekql nseslsqwtn pstmanakvk lsipkfkvek midpkaclen lglkhifsed |
| 301 | tsdfsgmset kgvalsnvih kvcleitedg gdsievpgar ilqhkdelna dhpfiyiirh |
| 361 | nktrniiffg kfcsp. |

In some embodiments, a therapeutic agent comprises a full-length maspin comprising at least 60% sequence identity (e.g., 60%, 65%, 70%, 75%, 80%, 85%, 90%, 91%, 92%, 93%, 94%,95%, 96%, 97%, 98%, 99%, 100%, or any ranges therebetween) to SEQ ID NO: 1. In some embodiments, a therapeutic agent comprises a full-length maspin comprising at least 60% sequence similarity (e.g., conservative similarity, semi-conservative similarity, etc.) to SEQ ID NO: 1 (e.g., 60%, 65%, 70%, 75%, 80%, 85%, 90%, 91%, 92%, 93%, 94%,95%, 96%, 97%, 98%, 99%, 100%, or any ranges therebetween). In some embodiments, a full-length maspin is an artificial maspin having less than 100% sequence identity with a natural maspin (e.g., human maspin, all natural maspins, etc.).

In some embodiments, a therapeutic agent within the scope herein is a maspin fragment, for example, comprising a peptide of 5, 6, 7, 8, 9, 10, 11, 12, 13, 14, 15, 16, 17, 18, 19, 20, 21, 22, 23, 24, or 25 (or ranges therebetween) consecutive amino acids of wild-type maspin (SEQ ID NO: 1). In some embodiments, a therapeutic agent comprises a maspin peptide comprising at least 60% sequence identity (e.g., 60%, 65%, 70%, 75%, 80%, 85%, 90%, 91%, 92%, 93%, 94%,95%, 96%, 97%, 98%, 99%, 100%, or any ranges therebetween) to 5, 6, 7, 8, 9, 10, 11, 12, 13, 14, 15, 16, 17, 18, 19, 20, 21, 22, 23, 24, or 25 (or ranges therebetween) consecutive amino acids of wild-type maspin (SEQ ID NO: 1). In some embodiments, a therapeutic agent comprises a maspin peptide comprising at least 60% (e.g., 60%, 65%, 70%, 75%, 80%, 85%, 90%, 91%, 92%, 93%, 94%,95%, 96%, 97%, 98%, 99%, 100%, or any ranges therebetween) sequence similarity (e.g., conservative similarity, semi-conservative similarity, etc.) to 5, 6, 7, 8, 9, 10, 11, 12, 13, 14, 15, 16, 17, 18, 19, 20, 21, 22, 23, 24, or 25 (or ranges therebetween) consecutive amino acids of wild-type maspin (SEQ ID NO: 1). In some embodiments, maspin peptide is an artificial maspin peptide having less than 100% sequence identity with a natural maspin (e.g., human maspin, all natural maspins, etc.).

In some embodiments, a therapeutic agent within the scope herein is an enhancer of maspin expression (e.g., maspin expression in osteoblast cells). In some embodiments, such an agent inhibits osteoclastogenesis and/or osteoclast activity by increasing the amount of maspin produced by a subject's own cells. In some embodiments, an enhancer of maspin expression is a small molecule, peptide, protein, nucleic acid, etc. In some embodiments, an enhancer of maspin expression is a protein (e.g., growth factor, transcription factor, etc.) or active peptide fragment thereof that that utilizes the expression machinery of a cell to scale-up expression of maspin. In some embodiments, an enhancer of maspin expression is a nucleic acid encoding maspin, to be expressed by the cells of the subject. In some embodiments, an enhancer of maspin expression alters the genomic DNA of the cell (e.g., osteoblasts of a subject) to increase maspin expression (e.g., by Cas/CRISPR).

In some embodiments, a maspin peptide that finds use in inhibiting osteoclastogenesis and/or osteoclast activity is one described in the literature for other purposes (e.g., treatment of cancer); for example, the peptides described in: U.S. Pat. No. 8,791,233; WO 2013/176667; WO 2007/095583; U.S. Pat. No. 9,169,294; U.S. Pat. No. 5,905,023; incorporated by reference in their entireties. In some embodiments, a maspin peptide that finds use in inhibiting osteoclastogenesis and/or osteoclast activity is a fragment of a maspin sequence described in the aforementioned patents and applications. In some embodiments, a maspin peptide that finds use in inhibiting osteoclastogenesis and/or osteoclast activity comprises at least 60% (e.g., 60%, 65%, 70%, 75%, 80%, 85%, 90%, 95%, 100%, or ranges therebetween) sequence identity and/or sequence similarity with all or a portion of a maspin sequence described in the aforementioned patents and applications.

In some embodiments, a therapeutic agent within the scope herein is an enhancer of maspin activity. In some embodiments, an enhancer of maspin expression is a small molecule, peptide, protein, antibody, etc. In some embodiments, an enhancer of maspin expression binds to an inhibitor of maspin activity and prevents its association with maspin and/or its inhibitory effect on maspin. In some embodiments, an enhancer of maspin expression binds to maspin and enhances its activity.

In some embodiments, cells expressing (e.g., overexpressing) maspin are provided to a subject (e.g., locally to a site in need of bone repair (e.g., due to injury, surgery, disease, etc.). In some embodiments, such cells are provided with a support matrix (e.g., extracellular matrix, synthetic extracellular matrix, etc.) to facilitate attachment and/or growth of the cells at the treatment site.

As discussed herein, in some embodiments, maspin peptides and/or proteins are provided having variations from natural maspin and/or from the sequences described herein. Embodiments are not limited by specific sequences and/or substitutions described herein. In some embodiments, peptides meeting limitations described herein (e.g., inhibiting osteoclastogenesis and/or osteoclast activity, etc.) and having substitutions not explicitly described are within the scope of embodiments herein. In some embodiments, the maspin peptides and/or polypeptides described herein are further modified (e.g., substitution, deletion, or addition of standard amino acids; chemical modification; etc.). Modifications that are understood in the field include N-terminal modification, C-terminal modification (which protects the peptide from proteolytic degradation), alkylation of amide groups, hydrocarbon “stapling” (e.g., to stabilize active conformations). In some embodiments, the maspin peptides and/or polypeptides described herein may be modified by conservative or semi conservative residue substitutions. In some embodiments, such substitutions provide subtle changes while preserving the local environment of the residue. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C₁-C₄ alkyl. Furthermore, one or more side groups, or terminal groups, may be protected by protective groups known to the ordinarily-skilled peptide chemist. The a-carbon of an amino acid may be mono- or dimethylated.

In some embodiments, any embodiments described herein may comprise peptidomimetics corresponding to the maspin peptides and/or polypeptides described herein with various modifications that are understood in the field. In some embodiments, residues in the maspin sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally-occurring amino acids other than the standard 20 amino acids) are substituted in order to achieve desired properties. In some embodiments, residues having a side chain that is positively charged under physiological conditions, or residues where a positively-charged side chain is desired, are substituted with a residue including, but not limited to: lysine, homolysine, δ-hydroxylysine, homoarginine, 2,4-diaminobutyric acid, 3-homoarginine, D-arginine, arginal (—COOH in arginine is replaced by —CHO), 2-amino-3-guanidinopropionic acid, nitroarginine (N(G)-nitroarginine), nitrosoarginine (N(G)-nitrosoarginine), methylarginine (N-methyl-arginine), ε-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine. A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges.

In some embodiments, residues having a side chain that is neutral/polar under physiological conditions, or residues where a neutral side chain is desired, are substituted with a reside including, but not limited to: asparagine, cysteine, glutamine, serine, threonine, tyrosine, citrulline, N-methylserine, homoserine, allo-threonine and 3,5-dinitro-tyrosine, and β-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, β-alanine, 1-aminocyclohexylcarboxylic acid, N-methylisoleucine, norleucine, norvaline, and N-methylvaline.

In some embodiments, maspin peptide and polypeptides are isolated and/or purified (or substantially isolated and/or substantially purified). Accordingly, in such embodiments, maspin peptides and/or polypeptides are provided in substantially isolated form. In some embodiments, maspin peptides and/or polypeptides are isolated from other peptides and/or polypeptides as a result of solid phase peptide synthesis, for example. Alternatively, maspin peptides and/or polypeptides can be substantially isolated from other proteins after cell lysis from recombinant production. Standard methods of protein purification (e.g., HPLC) can be employed to substantially purify maspin peptides and/or polypeptides.

In some embodiments, maspin peptides and/or polypeptides may be formulated in a number of different formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryprotectants (e.g., sugars such as trehalose), etc. The form of such preparations can be solutions, gels, etc. In some embodiments, maspin peptides and/or polypeptides are prepared in lyophilized form. Moreover, such preparations can include other desired agents, such as small molecules or other peptides, polypeptides or proteins. Indeed, such a preparation comprising a mixture of different embodiments of the peptides and/or polypeptides described here may be provided.

In some embodiments, provided herein are peptidomimetic versions of the peptide sequences described herein or variants thereof. In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Chapter 14 of “Drug Design and Development”, Krogsgaard, Larsen, Liljefors and Madsen (Eds) 1996, Horwood Acad. Publishers provides a general discussion of techniques for the design and synthesis of peptidomimetics and is herein incorporated by reference in its entirety. Suitable amide bond surrogates include, but are not limited to: N-alkylation (Schmidt, R. et al., Int. J. Peptide Protein Res., 1995, 46,47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. 0. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methyleneamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J.Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g. borane or a hydride reagent such as lithium aluminum-hydride); such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142; herein incorporated by reference in its entirety.

In some embodiments, maspin peptides and/or polypeptides are treated or conditioned prior to use. For example, maspin peptides and/or polypeptides may be incubated with a delivery vehicle, such as lipoproteins, nanoparticles, liposomes, etc., prior to use.

In some embodiments, the maspin peptides and/or polypeptides described herein are provided as fusions with other peptides or polypeptides. Such fusions may be expressed from a recombinant DNA which encodes the maspin peptides and/or polypeptides and the additional peptide/polypeptide or may be formed by chemical synthesis. For instance, the fusion may comprise a maspin peptide and/or polypeptide and an enzyme of interest, a luciferase, RNasin or RNase, and/or a channel protein (e.g., ion channel protein), a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, selectable marker protein, nucleic acid binding protein, extracellular matrix protein, secreted protein, receptor ligand, serum protein, a protein with reactive cysteines, a transporter protein, a targeting sequence (e.g., a myristylation sequence), a mitochondrial localization sequence, or a nuclear localization sequence. The additional peptide/polypeptide may be fused to the N-terminus and/or the C-terminus of the maspin peptide and/or polypeptide. In one embodiment, the fusion protein comprises a first peptide/polypeptide at the N-terminus and another (different) peptide/polypeptide at the C-terminus of the maspin peptide and/or polypeptide. Optionally, the elements in the fusion are separated by a connector sequence, e.g., preferably one having at least 2 amino acid residues, such as one having 13 and up to 40 or 50 amino acid residues. The presence of a connector sequence in a fusion protein of the invention does not substantially alter the function of either element (e.g., maspin peptide and/or polypeptide) in the fusion relative to the function of each individual element, likely due to the connector sequence providing flexibility (autonomy) for each element in the fusion. In certain embodiment, the connector sequence is a sequence recognized by an enzyme or is photocleavable. For example, the connector sequence may include a protease recognition site.

In some embodiments, provided herein are pharmaceutical compositions comprising the therapeutic agents (e.g., maspin peptides and/or polypeptides, enahncers of maspin expression or activity, inhibitors of maspin inhibitors, etc.) and a pharmaceutically acceptable carrier. Any carrier which can supply an active agent is a suitable carrier, and such carriers are well known in the art. In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intrasternal injection, or infusion) (e.g., as sterile injectable aqueous or non-aqueous solutions or suspensions)), nasally (including administration to the nasal membranes, such as by inhalation spray), topically (such as in the form of a cream or ointment), transdermally (such as by transdermal patch), or rectally (such as in the form of suppositories), etc. In some embodiments, provided herein are methods for treating patients suffering from (or at risk of) a bone disorder or injury and/or in need of treatment (or preventative therapy). In some embodiments, a pharmaceutical composition comprising at least one therapeutic agent described herein is delivered to such a patient in an amount and at a location sufficient to treat the condition. In some embodiments, pharmaceutical compositions are delivered to the patient systemically or locally, and it will be within the ordinary skill of the medical professional treating such patient to ascertain the most appropriate delivery route, time course, and dosage for treatment.

A pharmaceutical composition may be administered in the form which is formulated with a pharmaceutically acceptable carrier and optional excipients, adjuvants, etc. in accordance with good pharmaceutical practice. The pharmaceutical composition may be in the form of a solid, semi-solid or liquid dosage form: such as powder, solution, elixir, syrup, suspension, cream, drops, paste and spray. As those skilled in the art would recognize, depending on the chosen route of administration (e.g. pill, injection, etc.), the composition form is determined. In general, it is preferred to use a unit dosage form in order to achieve an easy and accurate administration of the active pharmaceutical peptide or polypeptide. In general, the therapeutically effective pharmaceutical compound is present in such a dosage form at a concentration level ranging from about 0.5% to about 99% by weight of the total composition, e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment.

The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

In some embodiments, pharmaceutical compositions (e.g., comprising maspin peptides and/or polypeptide, enhancers of maspin expression, level, and/or activity, inhibitors or maspin inhibitors, etc.) are co-administered (concurrently or in series) with one or more additional therapeutic agents. Additional therapeutic agents may comprise an anti-RANKL monoclonal antibody and/or bisphosphate. In some embodiments, co-administered agents are co-formulated. In other embodiments, the agents are separately formulated. Co-administration may occur concurrently or sequentially. For concurrent administration, the agents may be co-formulated or separately formulated. In some embodiments, the agents are administered by the same route or by separate routes of administration. For sequential administration, any suitable time lapse may occur between administrations, for example, 1 minute, 2 minutes, 5 minutes, 10 minutes, 30 minutes, 1 hour, 2 hours, 4 hours, 6 hours, 8 hours, 12 hours, 24 hours, 48 hours, 72 hours, 1 week, 2 weeks, 4 weeks, or more, or ranges there between. In some embodiments, pharmaceutical compositions (e.g., comprising maspin peptides and/or polypeptide, enhancers of maspin expression, level, and/or activity, inhibitors or maspin inhibitors, etc.) are provided as part of a kit.

All publications and patents listed below and/or provided herein are incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method of treating a bone disorder in a subject comprising administering to the subject an agent that enhances maspin expression, level, and/or activity within the subject.

2. The method of claim 1, wherein the agent is formulated in a pharmaceutical composition.

3. The method of claim 1, wherein the agent is a maspin peptide or polypeptide comprising at least 60% sequence identity to all or a portion of SEQ ID NO: 1.

4. The method of claim 1, wherein the agent interacts with the cellular machinery to increase expression of maspin within the cells of the subject.

5. The method of claim 4, wherein expression is increased within osteoblast cells.

6. The method of claim 1, wherein the agent is a nucleic acid.

7. The method of claim 6, wherein the nucleic acid encodes a maspin peptide or polypeptide and sequences to facilitate expression within the cells of the subject.

8. The method of claim 6, wherein the nucleic acid inhibits expression of an inhibitor of maspin activity or expression by antisense or RNA interference.

9. The method of claim 6, wherein the nucleic acid alters the genomic DNA of the cells of the subject to enhance expression of the subject's own maspin.

10. The method of claim 1, wherein the bone disorder is selected from the group consisting of osteopenia, osteoporosis, osteomalacia, rachitis, osteitis fibrosa, aplastic bone diseases, metabolic bone diseases, osteolysis, leucopenia, bone malformation, hypercalcemia, and nerve compression syndrome.

11. The method of claim 1, wherein an agent is administered locally to a treatment site or systemically to the subject.

12. A method of facilitating bone growth in a subject comprising increasing the level of maspin and/or maspin-based peptides or polypeptides within the subject.

13. The method of claim 12, wherein bone growth is facilitated by inhibiting osteoclastogenesis and/or osteoclast activity.

14. The method of claim 13, wherein bone growth is facilitated by reducing the rate of one resorption.

15. The method of claim 12, wherein the level of maspin and/or maspin-based peptides or polypeptides in increased within the subject by administering maspin and/or maspin-based peptides or polypeptides to the subject.

16. The method of claim 12, wherein the level of maspin and/or maspin-based peptides or polypeptides in increased within the subject by enhancing expression of endogenous maspin within the subject's cells.

17. The method of claim 12, wherein the level of maspin and/or maspin-based peptides or polypeptides in increased within the subject by administering cells to a subject that express maspin and/or maspin-based peptides or polypeptides.

18. A pharmaceutical composition comprising an active agent that enhances expression, level, or activity of masipin within a subject.

19. (canceled)


SEQUENCE LISTING (1 sequence)

SEQ ID NO: 1 - protein, 375 aa, Homo sapiens
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP